(19) United States
(12) Patent Application Publication
(10) Pub. No.: US 2017/0151171 A1
(43) Pub. Date: Jun. 1, 2017

(54) COSMETIC COMPOSITION FOR ANTI-AGING

(71) Applicants: Cosmax USA, Corp., Solon, OH (US); COSMAX, INC., Hwaseong-si (KR); Genesis Pure, Inc., Frisco, TX (US)

(72) Inventors: Jeongho An, Ridgewood, NJ (US); Alyssa Misoo Kim, Fort Lee, NJ (US); SO MI JEON, Seongnam-si (KR); Youn Hwa Nho, Daegu (KR); Minji Kim, Suwon-si (KR)

(21) Appl. No.: 15/052,223

(22) Filed: Feb. 24, 2016

(30) Foreign Application Priority Data
Nov. 26, 2015 (KR) 10-2015-0166362

(51) Int. Cl. A61K 8/99; A61K 8/42; A61K 8/98; A61K 8/97; A61Q 19/08; A61Q 19/00

(52) U.S. Cl. CPC A61K 8/99; A61Q 19/08; A61Q 19/007; A61K 8/987; A61K 8/97; A61K 8/42

(57) ABSTRACT

Provided is a cosmetic composition for anti-aging, which contains a spider web extract, a Pseudoalteromonas extract, a ginseng callus extract and a heptapeptide as active ingredients. The cosmetic composition for anti-aging exhibits superior antioxidant effect and is able to exhibit anti-aging effect by improving skin wrinkles and skin elasticity, enhancing skin-moisturizing ability and improving skin barrier function.

### CROSS-REFERENCE TO RELATED APPLICATIONS

This application claims, under 35 U.S.C. §119, the priority of Korean Patent Application No. 10-2015-0166362, filed on Nov. 26, 2015, in the Korean Intellectual Property Office, the disclosure of which is incorporated herein by reference in its entirety.

### TECHNICAL FIELD

The present disclosure relates to a cosmetic composition for anti-aging, more particularly to a cosmetic composition for anti-aging, which contains a spider web extract, a Pseudoalteromonas extract, a ginseng callus extract and a heptapeptide as active ingredients.

### BACKGROUND

Skin is one of the important human organs, as a protective barrier involved in body temperature regulation, barrier function and various physiological functions. Skin aging occurs largely via two processes, which are intrinsic aging occurring with aging regardless of environmental factors and photoaging caused by prolonged exposure to environmental factors such as UV. Physiological changes to skin caused by aging include thinning of the epidermis, dermis and subcutaneous tissue, decreased water content and skin dryness and various skin lesions such as pigmentation. In particular, reactive oxygen species generated due to excessive fatigue and stress, prolonged exposure to UV, etc. are the main cause of skin wrinkles and aging because they facilitate the synthesis of proteinases (MMPs) that degrade proteins such as collagen, elastin, etc. and decrease the elasticity of the dermis layer. Therefore, a lot of studies are underway on the protection and anti-aging of skin in the cosmetic field with regard to wrinkle formation, decrease in elasticity, pigmentation, change in skin texture, skin dryness, etc.

The stratum corneum, which is the outermost layer of the skin, not only protects our body but also regulates the water content of the skin by acting as a barrier. Natural moisturizing factors (NMFs) present in the cells of the human stratum corneum regulate the water content of the skin. Their functions decline gradually as the skin ages. Recently, many cases are reported where the skin surface becomes dry and skin elasticity decreases due to the lack of NMFs caused by extrinsic factors such as stress, UV, etc. Because decreased skin elasticity leads to wrinkle formation, an anti-aging effect may be achieved by maintaining the skin-moisturizing ability.

Aquaporins are proteins from a family of major intrinsic proteins which are present in all cell membranes and increase water absorption by selectively passing water molecules only. There are 13 known types of aquaporins in mammals. Among them, aquaporin 3 is expressed in keratinocytes and the absorption and passage of water and glycerol mediated by aquaporin 3 increase the water content and elasticity of skin by preventing water loss.

Recently, a lot of methods are studied to prevent skin aging. As a representative example, as the development of plant calluses having various physiological activities becomes easy due to the development of the plant culture technology based on the totipotency of plants, researches are actively underway in this regard. Meanwhile, the advancement in biotechnology has made the synthesis of various peptides involved in wrinkle improvement and anti-aging easier and attempts are made to deliver those various peptides more effectively to the skin through various systems as their in-vivo mechanisms are elucidated more elaborately and accurately. In addition, studies are carried out on the protective and defensive mechanisms of animals, plants and microorganisms living in uninhabitable or extreme environments such as the Arctic or Antarctic for application to cosmetics, and mechanisms for effectively providing such various effects to skin are also being developed actively.

Korean Patent Registration No. 1565542, which relates to a cosmetic composition for skin lifting and anti-aging, containing a spider web extract, describes that a cosmetic composition containing a spider web hydrolysate, obtained by hydrolyzing a spider web peptide or a spider web through fermentation with Laetiporus miniatus, as an active ingredient exhibits superior skin-moisturizing effect, skin elasticity-enhancing effect and skin wrinkle-improving effect. However, an anti-aging composition containing a spider web extract, a Pseudoalteromonas extract, a ginseng callus extract and a heptapeptide at a specific ratio has never been known.

The inventors of the present disclosure have made efforts to overcome the problems of the prior art. As a result, they have found out that a cosmetic composition for anti-aging, which contains a spider web extract, a Pseudoalteromonas extract, a ginseng callus extract and a heptapeptide as active ingredients has superior antioxidant effect and is able to exhibit anti-aging effect by improving skin wrinkles and skin elasticity, enhancing skin-moisturizing ability and improving skin barrier function.

### SUMMARY

The present disclosure is directed to providing a cosmetic composition for anti-aging, which has superior antioxidant effect and exhibits anti-aging effect by improving skin wrinkles and skin elasticity, enhancing skin-moisturizing ability and improving skin barrier function.

In an aspect of the present disclosure, the present disclosure provides a cosmetic composition for anti-aging, which contains a spider web extract, a Pseudoalteromonas extract, a ginseng callus extract and a heptapeptide as active ingredients.

The spider web extract of the present disclosure is an extract obtained by hydrolyzing a spider web which rich in human amino acids with an acid or a protease. It exhibits excellent effect of providing nutrients from the natural amino acids contained therein and regenerating cells. Also, because the thread-like protein aggregates of the spider web act as a water trap and some amino acids (Glu and Gly) have hydrophilic properties, it can provide excellent moisturizing effect.

The Pseudoalteromonas extract of the present disclosure is an extract obtained by fermenting Pseudoalteromonas antarctica NF3 which lives in very low-temperature environments of the Antarctica. Because it contains a lot of antifreeze glycoproteins, it provides protection from transepidermal water loss (TEWL) even in cold weather. In addition, it aids in skin regeneration by facilitating collagen and elastin synthesis.

The ginseng callus extract of the present disclosure is an active ingredient extracted from a callus of Korean ginseng (Panax ginseng Cameyer) obtained through stem cell culturing. It promotes the synthesis of collagen types 1 and 3, inhibits the activity of MMP-1 and -9 and exhibits an effect of preventing and improving wrinkles by regenerating cells.

In the present disclosure, the heptapeptide is a synthetic peptide mimicking the natural peptides found in human skin tissues involved in collagen synthesis, immune response, wound healing, etc. For example, it may be specifically a heptapeptide bound to palmitic acid, more specifically copper palmitoyl heptapeptide-14 and/or heptapeptide-15 palmitate.

In the test examples of the present disclosure, X-50 Antiaging CC Solution containing copper palmitoyl heptapeptide-14 and heptapeptide-15 palmitate were used.

The X-50 Antiaging CC Solution containing copper palmitoyl heptapeptide-14 and heptapeptide-15 palmitate exhibits anti-aging effect by promoting the production of the mRNAs of the proteins constituting the extracellular matrix (ECM) such as elastin, laminin, collagen, etc. through the ITD (Intelligent Targeting Device) technology.

The inventors of the present disclosure have found out that a cosmetic composition containing the ingredients exhibiting skin-moisturizing effect, wrinkle- and elasticity-improving effect and skin-regenerating effect at a specific ratio provide better anti-aging effect than when the ingredients are used alone due to a synergic effect.

In the present disclosure, the spider web extract, the Pseudoalteromonas extract, the ginseng callus extract and the heptapeptide may be mixed in a weight ratio of 1-2:1-2:1-2:1-2, more specifically 2:2:1:1.

According to experiments conducted by the inventors of the present disclosure, better anti-aging effect was achieved when the four ingredients (the spider web extract, the Pseudoalteromonas extract, the ginseng callus extract and the heptapeptide) were used together as compared to when they were used alone, in combination of two (the Pseudoalteromonas extract and the ginseng callus extract) or in combination of three (the spider web extract, the Pseudoalteromonas extract and the ginseng callus extract). The best anti-aging effect was achieved when the four ingredients were mixed at a ratio of 2:2:1:1.

In the present disclosure, the cosmetic composition may exhibit anti-aging effect by improving skin wrinkles and elasticity.

According to experiments conducted by the inventors of the present disclosure, it was found out that the expression of fibrillin and procollagen as a measure of improvement in skin wrinkle- and elasticity-improving effect was increased when the four ingredients were used together (Examples 1-2), in particular when the four ingredients were mixed at a ratio of 2:2:1:1 (Example 2), as compared to when the ingredients were used alone (Comparative Examples 1-4), in combination of two (Comparative Example 5) or in combination of three (Comparative Example 6). Based on this result, it can be seen that the cosmetic composition of the present disclosure can exhibit anti-aging effect by increasing the expression of fibrillin which plays an important role in the elasticity of human skin and the expression of procollagen which plays an important role in skin wrinkles and thereby improving skin wrinkles and elasticity (see Test Examples 2-3).

In the present disclosure, the cosmetic composition may exhibit anti-aging effect by enhancing skin-moisturizing ability. There are many causes of skin aging. Among them, water loss may cause skin troubles such as wrinkling, drooping, crumbling, dryness, etc., which are directly related with skin aging. Accordingly, skin moisturization is necessary to prevent skin aging.

According to experiments conducted by the inventors of the present disclosure, it was found out that the expression of aquaporin as a measure of skin-moisturizing effect was increased when the four ingredients were used together (Examples 1-2) as compared to when the ingredients were used alone (Comparative Examples 1-4), in combination of two (Comparative Example 5) or in combination of three (Comparative Example 6). Based on this result, it can be seen that the cosmetic composition of the present disclosure can exhibit anti-aging effect by increasing the expression of aquaporin which increases water absorption by skin cells and thereby improving skin-moisturizing ability (see Test Example 5).

In the present disclosure, the cosmetic composition may exhibit anti-aging effect by improving skin barrier function.

The skin has barrier function in order to maintain homeostasis in the human body in response to environmental change. The barrier function is exerted mainly by the stratum corneum which is the outermost layer of skin. The stratum corneum, which is the skin's primary defensive barrier, may be damaged by physical stimulation or injury. Those with atopy or dry skin have insufficient or damaged barrier function of the stratum corneum. Such insufficient barrier function may accelerate skin aging.

According to experiments conducted by the inventors of the present disclosure, it was found out that the expression of aquaporin transglutaminase-1 as a measure of improvement in skin barrier function was increased when the four ingredients were used together (Examples 1-2) as compared to when the ingredients were used alone (Comparative Examples 1-4), in combination of two (Comparative Example 5) or in combination of three (Comparative Example 6). Based on this result, it can be seen that the cosmetic composition of the present disclosure can exhibit anti-aging effect by increasing the expression of transglutaminase-1 whose expression is increased during the differentiation of human keratinocytes in the epidermis to the stratum corneum and which plays a critical role in the formation of cornified envelope and thereby improving skin barrier function (see Test Example 4).

In the present disclosure, the cosmetic composition may be prepared into one or more formulation selected from a group consisting of a skin lotion, a skin softener, a skin toner, an astringent, a lotion, a milk lotion, a moisturizing lotion, a nourishing lotion, a massage cream, a nourishing cream, a moisturizing cream, a hand cream, a foundation, an essence, a nourishing essence, a pack, a soap, a cleansing foam, a cleansing lotion, a cleansing cream, a body lotion and a body cleanser.

The cosmetic composition for anti-aging of the present disclosure, which contains the spider web extract, the Pseudoalteromonas extract, the ginseng callus extract and the heptapeptide as active ingredients, may prevent skin aging very effectively by promoting the expression of the factor that improves skin elasticity, the protein that prevents skin wrinkles, the factor that enhances skin barrier function and the factor that increases water absorption.

### BRIEF DESCRIPTION OF DRAWINGS

FIG. 1 shows the result of Test Example 2 showing increased expression of fibrillin as an elasticity factor. (Comp. Ex.: Comparative Example, Ex.: Example)

FIG. 2 shows the result of Test Example 3 showing increased expression of procollagen as a protein that prevents skin wrinkles.

FIG. 3 shows the result of Test Example 4 showing increased expression of transglutaminase as a protein that enhances skin barrier function.

FIG. 4 shows the result of Test Example 5 showing increased expression of aquaporin as a skin-moisturizing factor.

FIG. 5 shows the result of Test Example 6 wherein skin-moisturizing ability was tested for 8 hours.

### DETAILED DESCRIPTION OF EMBODIMENTS

Hereinafter, the present disclosure will be described in more detail through examples. However, the following examples are for illustrative purposes only and the scope of the present disclosure is not limited by them.

### EXAMPLE 1

### Preparation of Ingredients

As a spider web extract (A) used in the present disclosure, Spider Protein (Coseed Biopharm Co., Ltd., Korea) extracted from sterilized spider web after removing impurities was used.

As a Pseudoalteromonas extract (B) used in the present disclosure, Antarcticine C (Lipotec, Spain) which is an aqueous glycoprotein solution extracted from Pseudoalteromonas antarctica NF3, a microorganism which lives in very low-temperature environments of the Antarctica, was used.

As a ginseng callus extract (C) used in the present disclosure, PhytoG Stem Cell-Korea M. Ginseng(S) (GFC Co., Ltd., Korea) extracted from a stem cell culture of Korean ginseng was used.

As copper palmitoyl heptapeptide-14 and heptapeptide-15 palmitate used in the present disclosure, a synthetic peptide X-50 Antiaging CC Solution (Infinitec, USA) was used.

### TEST EXAMPLE 1

### Antioxidant Effect—Free Radical Scavenging Activity

The free radical scavenging activity of the ingredients of Example 1 was investigated using 1,1-diphenyl-2-pycrylhydrazyl (DPPH) which exists as a relatively stable free radical and is commonly used to test free radical scavenging activity.

After preparing samples from various combinations of the ingredients, 1 mL of 2 mM DPPH in ethanol was added at a concentration of 300 ppm. After waiting for 30 minutes at 37° C. and transferring to a 96-well plate, absorbance was measured at 517 nm using an ELISA reader. The free radical scavenging activity (%) was calculated according to Equation 1. The result is shown in Table 1.

Free radical scavenging activity (%)=100−B/A×100 [Equation 1]

A: absorbance of sample-untreated control group

B: absorbance of sample-treated test group

TABLE 1

| | | Scavenging |
|---|---|---|
| | Ingredients | activity (%) |
| Comparative Example 1 | Spider Protein (A) | 19.6 |
| Comparative Example 2 | Antarcticine (B) | 10.9 |
| Comparative Example 3 | PhytoG Stem Cell-Korea M (C) | 21.9 |
| Comparative Example 4 | X-50 Antiaging (D) | 4.8 |
| Comparative Example 5 | B + C, 1:1 | 25.1 |
| Comparative Example 6 | A + B + C, 1:1:1 | 25.9 |
| Example 1 | A + B + C + D, 1:1:1:1 | 30.0 |
| Example 2 | A + B + C + D, 2:2:1:1 | 35.2 |

As seen from Table 1, although the ingredients showed free radical scavenging activity when they were used alone, synergic effect was observed when the ingredients were used together. The best scavenging activity was achieved when the four ingredients were used together. In particular, when the ingredients were mixed at a specific ratio (Example 2), significantly improved free radical scavenging activity was achieved as compared to when they were used in equal amounts (Example 1).

### TEST EXAMPLE 2

### Elasticity-Enhancing Effect—Expression of Fibrillin

It was investigated whether the eight combinations of the ingredients that showed superior free radical scavenging activity (Comparative Examples 1-6, Examples 1-2) increase the production of fibrillin which plays an important role in the elasticity of human skin.

Human fibroblasts were inoculated onto a 6-well cell culture dish at a density of 4×10⁵ cells/well and then cultured in a 5% CO₂ incubator at 37° C. for 24 hours. Subsequently, the adherent cells were further cultured for 24 hours after treating with the eight combinations of the ingredients. Then, RNA was isolated from the cells using TRIzol (RNA iso, Dakara, Japan) and then quantified at 260 nm using NanoDrop. Subsequently, cDNA was synthesized from 2 μg of RNA (C1000 Thermal Cycler, Bio-Rad, USA). Finally, the expression level of the fibrillin gene was evaluated by conducing real-time polymerase chain reaction (PCR) of the synthesized cDNA using a mixture of the template (primer) of fibrillin as a target protein and a cyanine dye (SYBR Green Supermix, Applied Biosystems, USA). The gene expression level was normalized to that of the β-actin gene.

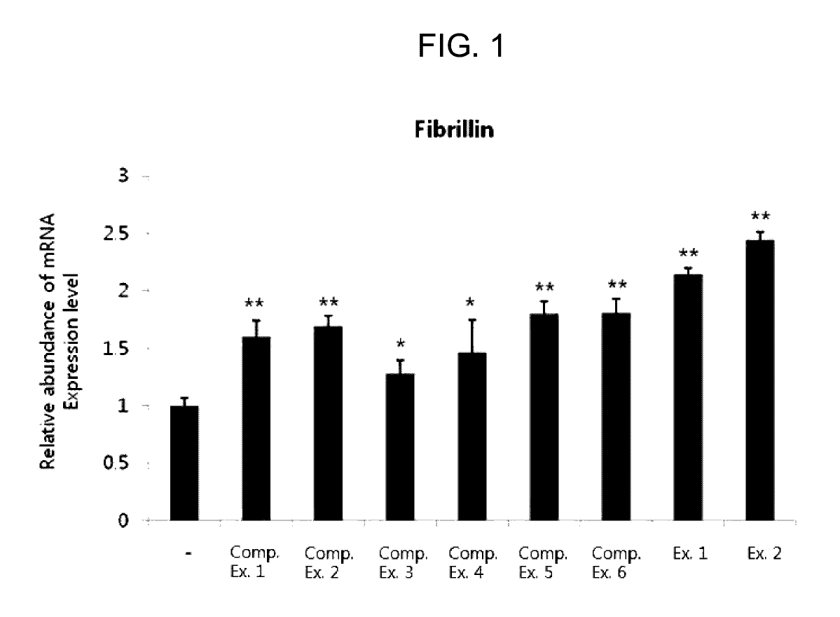

As seen from FIG. 1, it was found out that the eight combinations of the ingredients increased the expression of fibrillin. Among them, Example 1 and Example 2 wherein the spider web extract, the Pseudoalteromonas extract, the ginseng callus extract and the heptapeptide were used together showed the highest expression level of fibrillin.

### TEST EXAMPLE 3

### Wrinkle-Preventing Effect—Expression of Procollagen

It was investigated whether the eight combinations of the ingredients that showed superior free radical scavenging activity (Comparative Examples 1-6, Examples 1-2) increase the production of collagen which plays an important role in wrinkling in human skin.

Human fibroblasts were inoculated onto a 6-well cell culture dish at a density of 4×10⁵ cells/well and then cultured in a 5% CO₂ incubator at 37° C. for 24 hours. Subsequently, the cells were further cultured for 24 hours after irradiating 15 mJ of UVB and treating with the eight combinations of the ingredients. Then, RNA was isolated from the cells using TRIzol (RNA iso, Dakara, Japan) and then quantified at 260 nm using NanoDrop. Subsequently, cDNA was synthesized from 2 μg of RNA (C1000 Thermal Cycler, Bio-Rad, USA). Finally, the expression level of the type 1 procollagen gene was evaluated by conducing real-time polymerase chain reaction (PCR) of the synthesized cDNA using a mixture of the template (primer) of procollagen as a target protein and a cyanine dye (SYBR Green Supermix, Applied Biosystems, USA). The gene expression level was normalized to that of the β-actin gene.

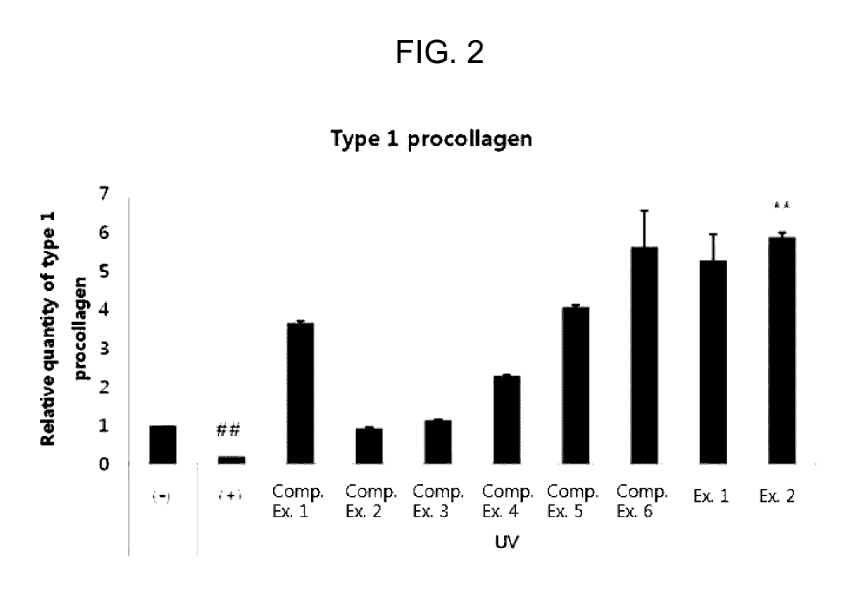

As seen from FIG. 2, it was found out that the eight combinations of the ingredients increased the expression of type 1 procollagen. Among them, Example 1 and Example 2 wherein the spider web extract, the Pseudoalteromonas extract, the ginseng callus extract and the heptapeptide were used together showed the highest expression level of type 1 procollagen.

### TEST EXAMPLE 4

### Barrier Function-Restoring Effect—Expression of Transglutaminase

It was investigated whether the eight combinations of the ingredients that (Comparative Examples 1-6, Examples 1-2) restore skin barrier function through expression of the transglutaminase 1 gene whose expression is increased during the differentiation of human keratinocytes in the epidermis to the stratum corneum and which plays a critical role in the formation of cornified envelope.

Human keratinocytes were inoculated onto a 6-well cell culture dish at a density of 4×10⁵ cells/well and then cultured in a 5% CO₂ incubator at 37° C. for 24 hours. Subsequently, the cells were further cultured for 24 hours after treating with the eight combinations of the ingredients. Then, RNA was isolated from the cells using TRIzol (RNA iso, Dakara, Japan) and then quantified at 260 nm using NanoDrop. Subsequently, cDNA was synthesized from 2 μg of RNA (C1000 Thermal Cycler, Bio-Rad, USA). Finally, the expression level of the transglutaminase 1 gene was evaluated by conducing real-time polymerase chain reaction (PCR) of the synthesized cDNA using a mixture of the template (primer) of transglutaminase 1 as a target protein and a cyanine dye (SYBR Green Supermix, Applied Biosystems, USA). The gene expression level was normalized to that of the β-actin gene.

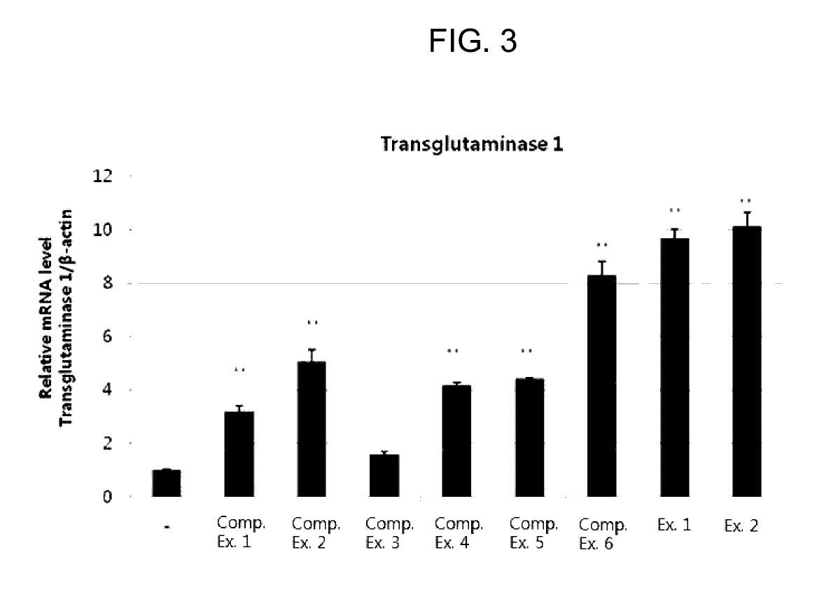

As seen from FIG. 3, it was found out that the eight combinations of the ingredients increased the expression of transglutaminase 1. Among them, Example 1 and Example 2 wherein the spider web extract, the Pseudoalteromonas extract, the ginseng callus extract and the heptapeptide were used together showed the highest expression level of transglutaminase 1.

### TEST EXAMPLE 5

### Moisturizing Effect—Expression of Aquaporin

It was investigated whether the eight combinations of the ingredients that (Comparative Examples 1-6, Examples 1-2) exhibit moisturizing effect by increasing the expression of aquaporin 3 which increases water absorption by skin cells.

Human keratinocytes were inoculated onto a 6-well cell culture dish at a density of 4×10⁵ cells/well and then cultured in a 5% CO₂ incubator at 37° C. for 24 hours. Subsequently, the cells were further cultured for 24 hours after treating with the eight combinations of the ingredients or 1 ppm retinol as a positive control. Then, RNA was isolated from the cells using TRIzol (RNA iso, Dakara, Japan) and then quantified at 260 nm using NanoDrop. Subsequently, cDNA was synthesized from 2 μg of RNA (C1000 Thermal Cycler, Bio-Rad, USA). Finally, the expression level of the aquaporin 3 gene was evaluated by conducing real-time polymerase chain reaction (PCR) of the synthesized cDNA using a mixture of the template (primer) of aquaporin 3 as a target protein and a cyanine dye (SYBR Green Supermix, Applied Biosystems, USA). The gene expression level was normalized to that of the β-actin gene.

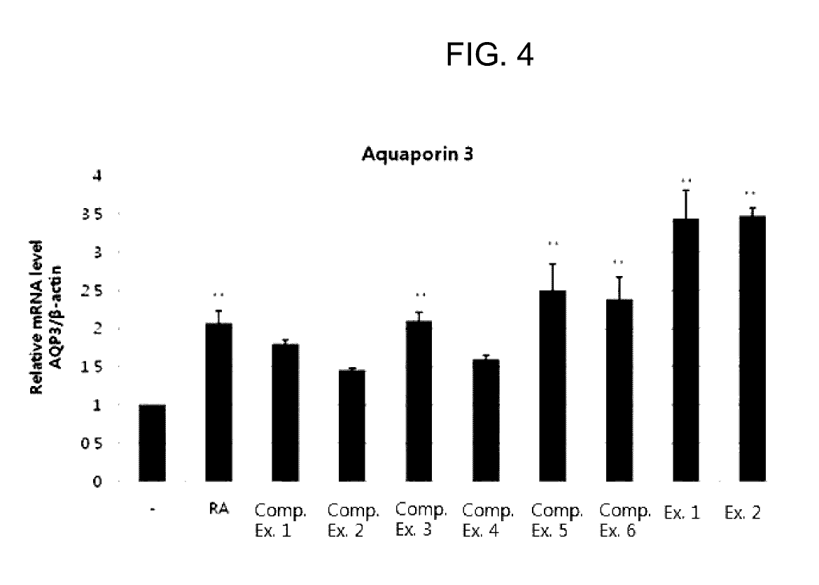

As seen from FIG. 4, it was found out that the eight combinations of the ingredients increased the expression of aquaporin 3. Among them, Example 1 and Example 2 wherein the spider web extract, the Pseudoalteromonas extract, the ginseng callus extract and the heptapeptide were used together showed the highest expression level of aquaporin 3, even higher than the positive control group treated with retinol.

### FORMULATION EXAMPLE 1

### Essence

An essence as a cosmetic formulation example containing the four ingredients of Example 2 is described in Table 2.

TABLE 2

| ingredients | Contents (unit: wt %) |
|---|---|
| Spider Protein | 1 |
| Antarcticine C | 1 |
| PhytoG Stem Cell-Korea M Ginseng | 0.5 |
| X-50 Antiaging CC Solution | 0.5 |
| Glycerin | 5 |
| Dimethicone | 13.5 |
| Disodium EDTA | 0.05 |
| Acrylate/C₁₀₋₃₀ alkyl acrylate crosspolymer | 0.12 |
| Carageenan | 0.2 |
| Ammonium acrylodimethyltaurate/VP copolymer | 0.2 |
| Preservative | 0.9 |
| Purified water | balance |
| Total | 100 |

### TEST EXAMPLE 6

### Skin-Moisturizing Ability (8 Hours)

The skin-moisturizing ability of a cosmetic containing the four ingredients of the present disclosure, Spider Protein, Antarcticine C, PhytoG Stem Cell-Korea M Ginseng and X-50 Antiaging CC Solution, was investigated as follows. Five healthy adult subjects aged 24.8 years on average were asked to uniformly apply 8 μL of the essence of Formulation Example 1 on test areas (2 cm×2 cm). Then, water content was measured 3 times using a corneometer (CK Electronic, Germany) immediately, 2 hours, 4 hours and 8 hours after the application.

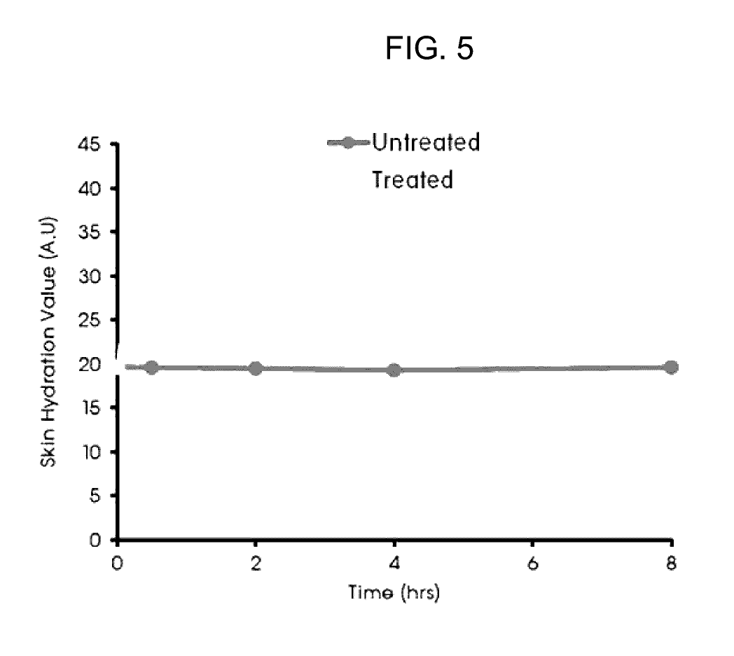

As seen from FIG. 5, the group treated with the essence showed increased water content for 8 hours as compared to the untreated group.

1. A cosmetic composition for anti-aging, which comprises a spider web extract, a Pseudoalteromonas extract, a ginseng callus stem cell extract, and a heptapeptide as active ingredients, wherein the heptapeptide is copper palmitoyl heptapeptide-14 and heptapeptide-15 palmitate.

2. (canceled)

3. The cosmetic composition for anti-aging according to claim 1, wherein the spider web extract, the Pseudoalteromonas extract, the ginseng callus stem cell extract and the heptapeptide are mixed in a weight ratio of 1-2:1-2:1-2:1-2.

4. The cosmetic composition for anti-aging according to claim 1, wherein the cosmetic composition exhibits anti-aging effect by improving skin wrinkles and elasticity.

5. The cosmetic composition for anti-aging according to claim 1, wherein the cosmetic composition exhibits anti-aging effect by enhancing skin-moisturizing ability.

6. The cosmetic composition for anti-aging according to claim 1, wherein the cosmetic composition exhibits anti-aging effect by improving skin barrier function.

7. The cosmetic composition for anti-aging according to claim 1, wherein the cosmetic composition is prepared into one or more formulation selected from a group consisting of a skin lotion, a skin softener, a skin toner, an astringent, a lotion, a milk lotion, a moisturizing lotion, a nourishing lotion, a massage cream, a nourishing cream, a moisturizing cream, a hand cream, a foundation, an essence, a nourishing essence, a pack, a soap, a cleansing foam, a cleansing lotion, a cleansing cream, a body lotion and a body cleanser.
